SEQUENCE LISTING (12 sequences)

SEQ ID NO: 1 - RNA, 22 nt, Homo sapiens
  source 1..22: mol_type = other RNA
aaaagctgggttgagagggcaa

SEQ ID NO: 2 - RNA, 23 nt, Homo sapiens
  source 1..23: mol_type = other RNA
tgaggggcagagagcgagacttt

SEQ ID NO: 3 - RNA, 24 nt, Homo sapiens
  source 1..24: mol_type = other RNA
ttaatgctaatcgtgataggggtt

SEQ ID NO: 4 - RNA, 21 nt, Homo sapiens
  source 1..21: mol_type = other RNA
tacagtactgtgataactgaa

SEQ ID NO: 5 - RNA, 22 nt, Homo sapiens
  source 1..22: mol_type = other RNA
tcagtgcactacagaactttgt

SEQ ID NO: 6 - RNA, 22 nt, Homo sapiens
  source 1..22: mol_type = other RNA
aaaagctgggttgagagggcga

SEQ ID NO: 7 - RNA, 22 nt, Homo sapiens
  source 1..22: mol_type = other RNA
ttcattcggctgtccagatgta

SEQ ID NO: 8 - RNA, 22 nt, Homo sapiens
  source 1..22: mol_type = other RNA
catcttaccggacagtgctgga

SEQ ID NO: 9 - RNA, 22 nt, Homo sapiens
  source 1..22: mol_type = other RNA
ctttcagtcggatgtttacagc

SEQ ID NO: 10 - RNA, 23 nt, Homo sapiens
  source 1..23: mol_type = other RNA
caacggaatcccaaaagcagctg

SEQ ID NO: 11 - RNA, 22 nt, Homo sapiens
  source 1..22: mol_type = other RNA
tgaggtagtaggttgtatagtt

SEQ ID NO: 12 - RNA, 22 nt, Homo sapiens
  source 1..22: mol_type = other RNA
tgaggtagtagattgtatagtt

(19) United States
(12) Patent Application Publication
(10) Pub. No.: US 2026/0159897 A1
(43) Pub. Date: Jun. 11, 2026

(54) EXOSOME-DERIVED microRNA BIOMARKERS ISOLATED FROM DUODENAL-PANCREATIC FLUID FOR PANCREATIC CANCER DIAGNOSIS AND USES THEREOF

(71) Applicants: THE ASAN FOUNDATION, Seoul (KR); UNIVERSITY OF ULSAN FOUNDATION FOR INDUSTRY COOPERATION, Ulsan (KR); ALPS MEDICAL CO., LTD, Seoul (KR)

(72) Inventors: Do Hyun PARK, Seoul (KR); Chang Hoon HA, Seoul (KR); Ce Hwan PARK, Seoul (KR)

(73) Assignees: THE ASAN FOUNDATION, Seoul (KR); UNIVERSITY OF ULSAN FOUNDATION FOR INDUSTRY COOPERATION, Ulsan (KR); ALPS MEDICAL CO., LTD, Seoul (KR)

(21) Appl. No.: 19/169,187

(22) Filed: Apr. 3, 2025

(30) Foreign Application Priority Data
Dec. 10, 2024 (KR) 10-2024-0182904

(51) Int. Cl. C12Q 1/6886

(52) U.S. Cl. CPC C12Q 1/6886; C12Q 2600/112; C12Q 2600/158; C12Q 2600/178

(57) ABSTRACT

Provided are exosome-derived microRNA biomarkers isolated from duodenal-pancreatic fluid for diagnosing pancreatic cancer, and uses thereof. Particularly, provided are a microRNA biomarker for diagnosing pancreatic cancer, a composition for diagnosing pancreatic cancer using the same, and a method for providing diagnostic information using the same. The microRNAs described herein demonstrate excellent diagnostic specificity and sensitivity for pancreatic cancer, and thus can be usefully employed in a non-invasive manner for early diagnosis of pancreatic cancer, as well as for predicting progression and treatment response of pancreatic cancer.

### CROSS-REFERENCE TO RELATED APPLICATION

This application is based on and claims priority under 35 USC § 119 to Korean Patent Application No. 10-2024-0182904, filed on Dec. 10, 2024, in the Korean Intellectual Property Office, the disclosure of which is incorporated by reference herein in its entirety.

### REFERENCE TO A SEQUENCE LISTING

In accordance with 37 CFR § 1.831-1835 and 37 CFR § 1.77 (b) (5), the specification makes reference to a Sequence Listing submitted electronically as a .xml file named “557571US_ST26.xml”. This .xml file was generated on Apr. 2, 2025 and is 11,639 bytes in size. The entire contents of the Sequence Listing are hereby incorporated by reference.

### BACKGROUND

### 1. Field

The present disclosure relates to a biomarker composition for diagnosing pancreatic cancer, which includes exosome-derived microRNA isolated from duodenal-pancreatic fluid as an active ingredient.

### 2. Description of the Related Art

Among the major diseases that commonly occur in modern people, research on treatment and diagnostic methods for cancer has primarily focused on lung cancer, liver cancer, and stomach cancer, which have high incidence rates. However, research on cancer types with relatively lower incidence rates, such as esophageal cancer, colorectal cancer, and pancreatic cancer, is still insufficient.

Among these, pancreatic cancer is one of the most fatal malignancies occurring in the gastrointestinal tract, typically showing no specific symptoms in its early stages but manifesting severe symptoms once systemic metastasis has occurred. The clinical symptoms of pancreatic cancer generally develop gradually, with loss of appetite and weight loss being the most common symptoms. Due to its substantially lower treatment success rate compared to other cancers, patients diagnosed with pancreatic cancer have an average survival period of approximately 14 months. A pancreatic cancer has a 1-4% five-year survival rate and a median survival period of approximately 5 months, showing the poorest prognosis among all human cancers.

To date, several anticancer agents, including 5-fluorouracil, gemcitabine, and nab-paclitaxel (Abraxane), have been reported to be effective against pancreatic cancer. However, the response rate to anticancer therapy is only approximately 15%, and cases of cancer metastasis to other organs have been reported as a side effect of chemotherapy in some patients. Therefore, there is an urgent need for a method for early diagnosis of pancreatic cancer to improve patient prognosis.

MicroRNAs (miRNAs) are small non-coding RNAs that regulate various physiological and developmental processes within cells by controlling the expression of target mRNAs. These miRNAs play an important role in gene expression regulation and have garnered attention as useful biomarkers for identifying the presence, progression, and prognosis of diseases through specific expression changes associated with specific disease states.

In particular, miRNAs in exosomes show characteristic changes in expression patterns under specific disease conditions. Reports commonly indicate that specific miRNAs are either overexpressed or downregulated in exosomes from cancer patients, and these miRNA profiles provide important information about not only the presence of cancer but also the cancer type, stage, and prognosis. Currently, exosomal miRNAs are being investigated as important biomarkers in gastrointestinal cancers, such as gastric cancer and colorectal cancer, and are being utilized as valuable indicators for predicting tumor progression, metastatic potential, and treatment response.

Furthermore, since exosomal miRNAs are protected from the external environment by the lipid bilayer membrane of exosomes, they exhibit resistance to degradation and can maintain stability in blood or other bodily fluids for extended periods. These characteristics suggest that exosomal miRNAs may serve as clinically reliable biomarkers. Additionally, exosomal miRNAs can be readily detected in blood, urine, saliva, or other bodily fluids, providing potential for non-invasive diagnostic methods that could replace invasive diagnostic procedures such as tissue biopsy. However, bodily fluids such as blood and urine contain exosomes circulating from the entire body rather than from a single organ, limiting their ability to provide disease specificity for a particular organ.

Accordingly, the present disclosure provides an exosome-derived microRNA biomarker isolated from duodenal-pancreatic fluid for diagnosing pancreatic cancer, and uses thereof.

### SUMMARY

One aspect of the present disclosure provides a biomarker composition for diagnosing pancreatic cancer, which includes exosome-derived miRNA isolated from duodenal-pancreatic fluid.

Another aspect of the present disclosure provides a composition for diagnosing pancreatic cancer, which includes a reagent capable of measuring the expression level of the biomarker.

Another aspect of the present disclosure provides a kit for diagnosing pancreatic cancer, which includes the composition for diagnosing pancreatic cancer as an active ingredient.

Another aspect of the present disclosure provides a method of diagnosing pancreatic cancer by measuring the expression level of the biomarker.

Another aspect of the present disclosure provides a biomarker composition for diagnosing pancreatic cancer, which includes at least one miRNA selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p.

Another aspect of the present disclosure provides a method for diagnosing pancreatic cancer, which comprises obtaining a biological sample from a subject, measuring the expression level of at least one miRNA selected from the group consisting of miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p in the biological sample, and diagnosing pancreatic cancer based on the expression level of the at least one miRNA.

In the present disclosure, the terms “miRNA”, “microRNA”, or “miR”, unless otherwise specified, refer to non-coding RNAs of 15-25 bases that are transcribed as hairpin-structured RNA precursors, cleaved by dsRNA-cleaving enzymes with RNase III activity, introduced into a protein complex called RISC, and involved in the translational repression of mRNA. In addition, the miRNAs used in the present disclosure include not only miRNAs having specific base sequences (or SEQ ID NOs), but also precursors (pre-miRNA and pri-miRNA) of these miRNAs, miRNAs having equivalent biological functions (for example, homologs or orthologs), variants including gene polymorphisms, and derivatives. These precursors, homologs, variants, and derivatives can be identified using miRBase release 22.1 (http://www.mirbase.org/) and may include miRNAs having base sequences that hybridize, under stringent conditions, to complements of the miRNAs. In addition, the miRNAs mentioned in the present disclosure may be gene products of miR genes, and such gene products may include mature miRNAs (for example, 15-25-base or 19-25-base non-coding RNAs involved in translational repression of mRNA as described above) or miRNA precursors (for example, pre-miRNAs or pri-miRNAs as described above).

Furthermore, the base sequences of the miRNAs mentioned in the present specification may be SEQ ID NOs: 1 to 12.

TABLE 1

| SEQ | | |
|---|---|---|
| ID NO. | miRNA name | Sequence (5′→3′) |
| 1 | miR-320b | AAAAGCUGGGUUGAGAGGGCAA |
| 2 | miR-423-5p | UGAGGGGCAGAGAGCGAGACUUU |
| 3 | miR-155-5p | UUAAUGCUAAUCGUGAUAGGGGUU |
| 4 | miR-101-3p | UACAGUACUGUGAUAACUGAA |
| 5 | miR-148a-3p | UCAGUGCACUACAGAACUUUGU |
| 6 | miR-320a-3p | AAAAGCUGGGUUGAGAGGGCGA |
| 7 | miR-1298-5p | UUCAUUCGGCUGUCCAGAUGUA |
| 8 | miR-200a-5p | CAUCUUACCGGACAGUGCUGGA |
| 9 | miR-30e-3p | CUUUCAGUCGGAUGUUUACAGC |
| 10 | miR-191-5p | CAACGGAAUCCCAAAAGCAGCUG |
| 11 | Let-7a-5p | UGAGGUAGUAGGUUGUAUAGUU |
| 12 | Let-7f-5p | UGAGGUAGUAGAUUGUAUAGUU |

In the present disclosure, the term “diagnosis” refers to verifying the existence or characteristics of a pathological condition, and for the purposes of the present disclosure, refers to verifying whether pancreatic cancer has developed or is likely to develop, and includes determining the susceptibility of a subject to pancreatic cancer or at least one symptom thereof, as well as therametrics (for example, monitoring a subject's condition for providing information on therapeutic efficacy), and so on. This also includes the primary diagnosis of a clinical condition or the diagnosis of a recurrent disease.

In the present disclosure, the term “biomarker” refers to an indicator capable of detecting changes in the body and includes substances that can verify whether a living organism is in a normal or pathological state or changes thereof. Such substances may include organic biomolecules, such as polypeptides, nucleic acids, lipids, glycolipids, glycoproteins, and sugars (monosaccharides, disaccharides, oligosaccharides, etc.).

In an embodiment, the miRNA may be preferably derived from exosomes isolated from duodenal-pancreatic fluid.

The term “exosome” or “exosom” refers to small vesicles with a membrane structure that are released when a multivesicular body-a vesicle formed during the maturation process of an endosome-fuses with the cell membrane, and may be extracellular vesicles (EVs). The released exosomes contain miRNAs and may have a diameter of about 30 nm to about 500 nm, about 30 nm to about 400 nm, about 30 nm to about 300 nm, about 30 nm to about 200 nm, about 50 nm to about 200 nm, about 50 nm to about 180 nm, or about 75 nm to about 180 nm.

In another embodiment, the pancreatic cancer may be pancreatic ductal adenocarcinoma, but is not limited thereto.

In another embodiment, the miRNAs described herein exhibit excellent diagnostic specificity and sensitivity for pancreatic cancer and may be usefully employed in a non-invasive manner to diagnose pancreatic cancer, as well as to predict progression and treatment response of pancreatic cancer (see FIGS. 10 and 11).

Another aspect of the present disclosure provides a composition for diagnosing pancreatic cancer, including a reagent capable of measuring the expression level of at least one miRNA selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p.

These miRNAs are as described in the section “biomarker composition for diagnosing pancreatic cancer” above.

The reagent capable of measuring the expression level may include the miRNA or a contiguous polynucleotide of the miRNA or a portion thereof, a polynucleotide complementary thereto, or a primer or probe that specifically binds thereto.

The primer refers to a short nucleic acid sequence with a free 3′ hydroxyl group, capable of base pairing with a complementary template and functioning as an initiation site for copying the template strand. Primers may initiate DNA synthesis in the presence of a suitable buffer solution and temperature, reagents for polymerization (i.e., DNA polymerase or reverse transcriptase), and four different nucleoside triphosphates. In the present disclosure, pancreatic cancer may be diagnosed by confirming the expression level through PCR amplification using sense and antisense primers that specifically bind to the at least one miRNA. The PCR conditions and the lengths of sense and antisense primers may be modified based on what is known in the art.

In addition, the probe refers to a labeled nucleic acid fragment, such as RNA or DNA, ranging from a few bases to several dozen bases in length. The probe may be prepared in the form of an oligonucleotide probe, single-stranded DNA probe, double-stranded DNA probe, or RNA probe. In the present disclosure, pancreatic cancer may be diagnosed by confirming the expression level through hybridization using a probe complementary to the at least one miRNA. The selection of a suitable probe and the hybridization conditions may be modified based on what is known in the art.

Furthermore, primers or probes may be suitably designed by those skilled in the art based on known sequences. For example, primers or probes may be chemically synthesized using a phosphoramidite solid-support method or other widely known methods. Such nucleic acid sequences may also be modified by various means known in the art. Non-limiting examples of such modifications include methylation, capping, substitution with one or more homologs of naturally occurring nucleotides, and modifications between nucleotides, for example, the introduction of uncharged linkages (e.g., methyl phosphonate, phosphotriester, phosphoramidate, carbamate, etc.) or charged linkages (e.g., phosphorothioate, phosphorodithioate, etc.).

The expression level of the miRNA may be measured by methods commonly used in the art, such as next-generation sequencing (NGS), reverse transcription polymerase chain reaction (RT-PCR), competitive RT-PCR, real-time RT-PCR, RNase protection assay (RPA), Northern blotting, or gene chips, among others, without being limited thereto.

Another aspect of the present disclosure provides a kit for diagnosing pancreatic cancer, which includes the aforementioned composition for diagnosing pancreatic cancer.

The kit may be at least one selected from an RT-PCR kit, a competitive RT-PCR kit, a real-time RT-PCR kit, a DNA chip kit, a microarray kit, a SAGE (Serial Analysis of Gene Expression) kit, and a protein chip kit, but is not limited thereto. For example, the kit may be a kit including components essential for performing RT-PCR. For example, such an RT-PCR kit may include test tubes or other suitable containers, reaction buffers (with various pH and magnesium concentrations), deoxynucleotides (dNTPs), dideoxynucleotides (ddNTPs), enzymes such as Taq polymerase and reverse transcriptase, DNase, RNase inhibitors, DEPC-treated water, and sterile water, in addition to primers specific to the miRNA genes. The kit may also include primer pairs specific to DNA, RNA, or miRNA used as quantitative controls. The kit of the present disclosure may include a kit for extracting nucleic acids (for example, total RNA), fluorescent materials for labeling, enzymes and media for nucleic acid amplification, and instructions for use. The kit of the present disclosure may be a device for measuring biomarkers for pancreatic cancer, wherein the nucleic acid is bound or attached to a solid support. Examples of materials for the solid support may include plastic, paper, glass, silicone, and the like, wherein plastic may be preferred due to its ease of processing. The shape of the solid support is not particularly limited and may be, for example, square, circular, rectangular, or film-shaped.

Another aspect of the present disclosure provides a method of providing information for diagnosing pancreatic cancer, the method including: (a) measuring, from a biological sample isolated from a subject, the expression level of at least one miRNA gene selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p; and

(b) comparing the expression level of the at least one miRNA gene selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p measured in step (a) with the expression level of the same miRNA genes measured in a biological sample obtained from a normal control.

In an embodiment, the biological sample may be at least one selected from tissue, cells, whole blood, blood, serum, saliva, sputum, cerebrospinal fluid, urine, and exosomes derived therefrom, and may be more preferably exosomes derived from duodenal-pancreatic fluid.

In another embodiment, the subject may be preferably a human who has, or is likely to develop, pancreatic cancer, but may also be a non-human primate (including chimpanzees), a pet (e.g., dog, cat), a livestock animal (e.g., cattle, horse, sheep, goat), or a rodent (e.g., mouse, rat), without being limited thereto.

The method may further include, without being limited to, step (c-1) of diagnosing pancreatic cancer when the expression level of at least one miRNA gene selected from miR-320b, miR-423-5p, miR-155-5p, miR-101-3p, miR-148a-3p, miR-320a-3p, miR-1298-5p, and miR-200a-5p, as measured in step (a), is higher than the expression level of the same miRNA gene measured in the biological sample obtained from the normal control in step (b).

The method may further include, without being limited to, step (c-2) of diagnosing pancreatic cancer when the expression level of at least one miRNA gene selected from miR-30e-3p, miR-191-5p, Let-7a-5p and Let-7f-5p, as measured in step (a) is lower than the expression level of the same miRNA gene measured in the biological sample obtained from the normal control in step (b).

In the aforementioned steps, if, in addition, the expression level of miR-155-5p or miR-423-5p gene is higher than the expression level of the same miRNA gene measured in the biological sample obtained from the normal control, stage 1 pancreatic cancer may be diagnosed; and if, in addition, the expression level of miR-200a-5p gene is higher than the expression level of the same miRNA gene measured in the biological sample obtained from the normal control, information for diagnosing end-stage pancreatic cancer may be provided.

In the aforementioned steps, if, in addition, the expression level of miR-30e-3p gene is lower than the expression level of the same miRNA gene measured in the biological sample obtained from the normal control, information for diagnosing end-stage pancreatic cancer may be provided.

In an embodiment, miRNAs specifically expressed in each stage of pancreatic cancer patients were analyzed based on fold change values relative to a control group; a candidate group of miRNAs (miR-155-5p, miR-423-5p) that showed characteristic expression changes in stage 1 pancreatic cancer patients was identified; and miRNAs (miR-101-3p, miR-148a-3p, miR-1298-5p) that were increased in all pancreatic cancer stages, as well as miRNAs (miR-200a-5p, miR-30e-3p) that were specifically altered in end-stage pancreatic cancer patients, were confirmed (see FIG. 9).

The expression level may be measured by one or more methods selected from next-generation sequencing (NGS), RT-PCR, real-time PCR, RNase protection assay (RPA), microarray, and Northern blotting; however, various known test methods may be suitably employed.

### BRIEF DESCRIPTION OF THE DRAWINGS

The above and other aspects, features, and advantages of certain embodiments of the disclosure will be more apparent from the following description taken in conjunction with the accompanying drawings in which:

FIG. 1 is a schematic diagram illustrating duodenal pancreatic duct and exosomes derived from pancreatic fluid;

FIGS. 2A and 2B are a view showing qualitative/quantitative analysis results of exosomes derived from duodenal-pancreatic fluid using NANOSIGHT;

FIG. 3 is a view comparing exosomes between control and patient groups based on exosome NANOSIGHT data;

FIG. 4 is a view showing results of heatmap clustering analysis using miRNA sequencing data from exosomes derived from duodenal-pancreatic fluid between control and pancreatic cancer groups;

FIG. 5 is a view showing heatmap expression analysis results of miRNAs showing the greatest increases and decreases in exosomes derived from duodenal-pancreatic fluid between control and pancreatic cancer groups;

FIG. 6 is a view showing results of a correlation matrix analysis based on sequencing data from individual control and pancreatic cancer groups;

FIG. 7 is a view showing results of 3D principal component analysis (PCA) using expression patterns from pancreatic cancer and control groups;

FIG. 8 is a view showing results of group analyses by TNM stages between control and pancreatic cancer groups;

FIG. 9 is a view showing results of Venn diagram analyses by TNM stages between control and pancreatic cancer groups;

FIGS. 10A and 10B are a view showing AUC analysis results for eight biomarkers showing increased expression in a pancreatic cancer group among the twelve biomarkers of the present disclosure;

FIG. 11 is a view showing the AUC analysis results for four biomarkers showing decreased expression in a pancreatic cancer group among the twelve biomarkers of the present disclosure; and

FIG. 12 is a schematic diagram showing a process of discovering miRNA biomarkers from duodenal-pancreatic fluid exosomes according to the present disclosure.

### DETAILED DESCRIPTION

The present disclosure will now be described in greater detail through the following examples. However, these examples are provided for illustrative purposes only and are not intended to limit the scope of the present disclosure.

### Example 1. Isolation of Exosomes Derived from Duodenal-Pancreatic Fluid and miRNAs Therein

The present disclosure was conducted with approval of the Institutional Review Board (IRB) of Seoul Asan Medical Center. Duodenal-pancreatic fluid was collected via endoscopy from 11 patients diagnosed with early-stage pancreatic ductal adenocarcinoma (PDAC), 9 patients diagnosed with end-stage PDAC, and 8 normal controls.

The normal control group was designated as “DC”, and each normal control was assigned a separate number (e.g., DC 005, 006, 007, 009, 010, 012, and 013). The pancreatic cancer patients were designated as “DP”, and each patient was assigned a separate number (e.g., DP 002, 003, 004, 005, 009, 010, 011, 012, 014, 017, 019, 021, 022, 023, 025, 026, 027, 024, 028, and 029).

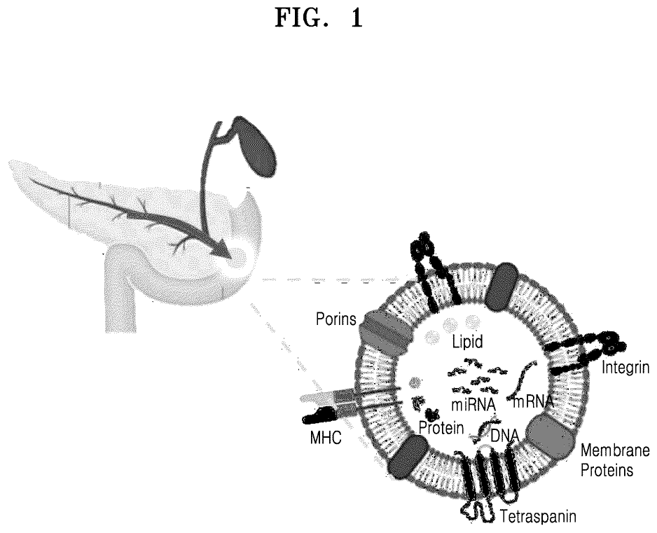

The duodenal-pancreatic fluid samples obtained from the normal controls and the pancreatic cancer patients were centrifuged at 12,000×g for 30 minutes at 4° C., and the supernatant was collected to remove cell debris. The separated supernatant was then subjected to centrifugation at 100,000×g for 1 hour in an ultracentrifuge to obtain the pellet (exosomes), which was washed with PBS and re-centrifuged at 100,000×g for 1 hour. The resulting exosomes (see FIG. 1) were resuspended in 20 μL of PBS, and miRNA was purified using an miRNeasy Micro Kit (Qiagen) according to the manufacturer's instructions.

### Example 2. Characterization of Exosomes Derived from Duodenal-Pancreatic Fluid

Exosomes isolated in Example 1 were analyzed for size distribution using a NANOSIGHT (NS300) instrument to determine their size and concentration. Data were interpreted using NTA 2.1 analytical software, and the exosome size distribution was plotted.

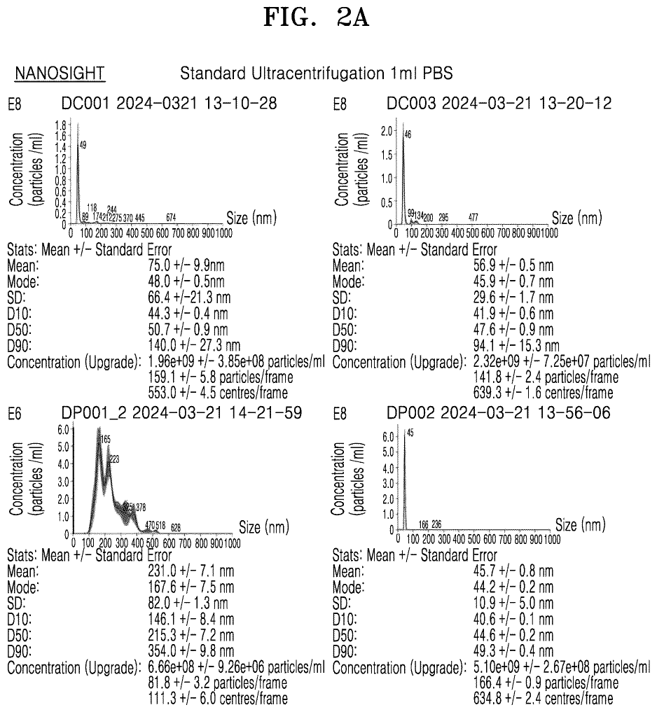

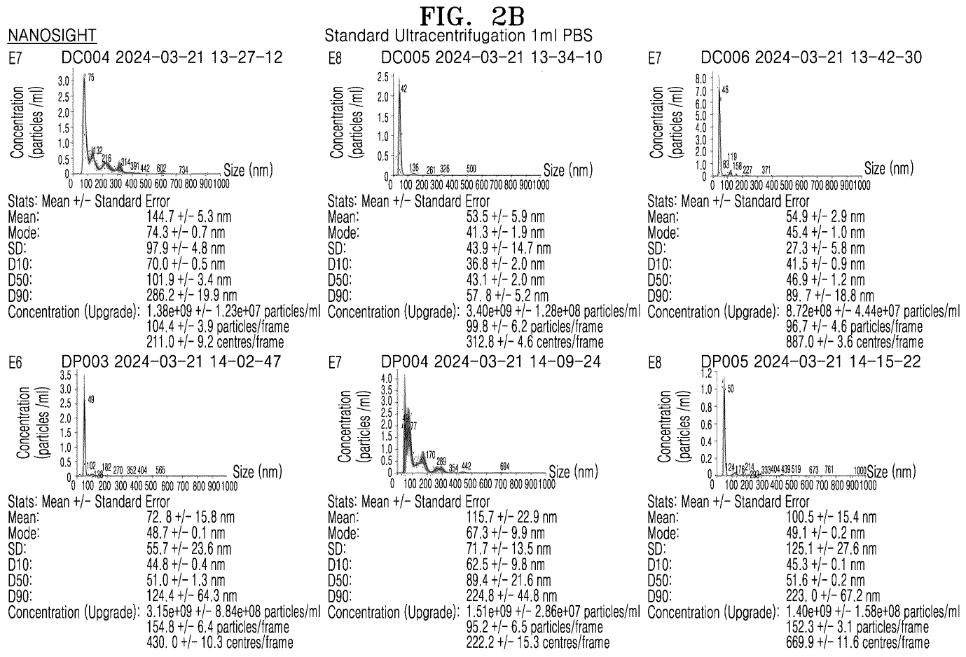

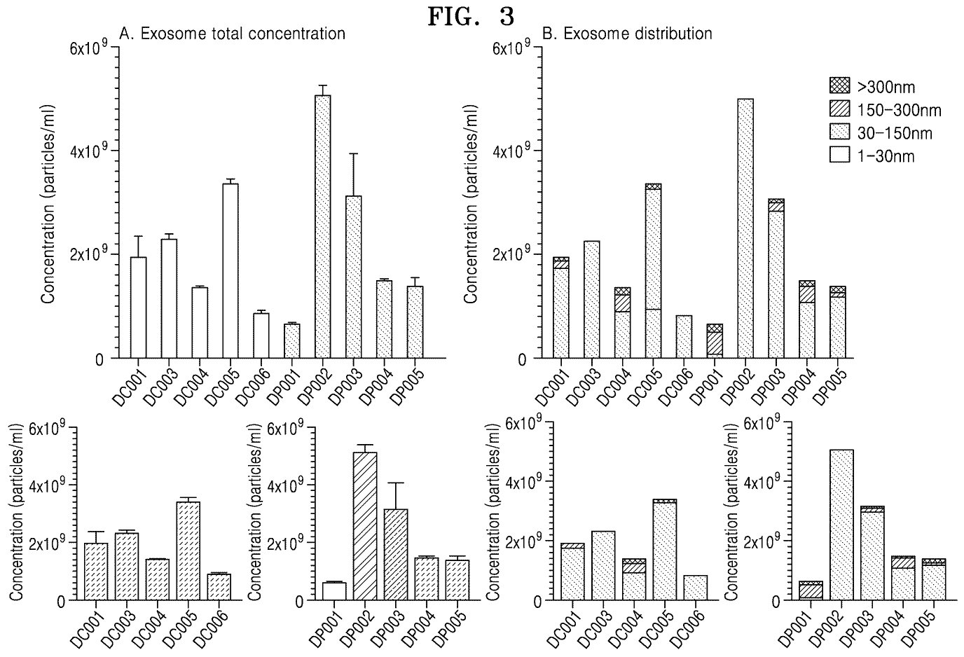

As shown in FIGS. 2 and 3, while there was a slight increase in the concentration of exosomes in the pancreatic cancer group compared to the control group, the exosomes isolated from both groups had sizes within the standard exosome range of 30 nm to 150 nm (see FIGS. 2 and 3).

### Example 3. Selection of Candidate Biomarkers in Exosomes Derived from Duodenal-Pancreatic Fluid

MiRNAs in exosomes derived from the duodenal-pancreatic fluid of eight control subjects and twenty pancreatic cancer patients, as described in Example 1, were analyzed.

Specifically, small-RNA sequencing was performed to initially determine the overall miRNA expression in exosomes derived from the duodenal-pancreatic fluid of the control subjects and pancreatic cancer patients, and statistical analyses were conducted on all the miRNAs.

The RNA quality was assessed using an Agilent 2100 Bioanalyzer (or TapeStation 4000 system) and an RNA 6000 Pico Chip (Agilent Technologies, Amstelveen, The Netherlands). The RNA quantification was performed using a NanoDrop 2000 Spectrophotometer system (Thermo Fisher Scientific, Waltham, MA, USA) or a Qubit system (Thermo Fisher Scientific Inc., USA).

Libraries were constructed for the control and experimental group RNAs using a NEBNext Multiplex Small RNA Library Prep Kit (New England BioLabs, Inc.) according to the manufacturer's instructions.

Specifically, total RNA from each sample was used to ligate adapters, followed by CDNA synthesis using adapter-specific primers and reverse transcriptase. Library amplification was performed by PCR, and purification was conducted using a QIAquick PCR Purification Kit (Qiagen, Inc., Germany) and polyacrylamide gel electrophoresis (PAGE). The yield and size distribution of the small RNA libraries were measured using a High-Sensitivity DNA Analysis Chip (Agilent Technologies, Inc., USA) with an Agilent 2100 Bioanalyzer. High-throughput sequencing was performed using a NextSeq550 system (Illumina, San Diego, CA, USA) with single-end 75 sequencing. The small RNA sequencing was conducted by E-biogen (Seoul, Republic of Korea).

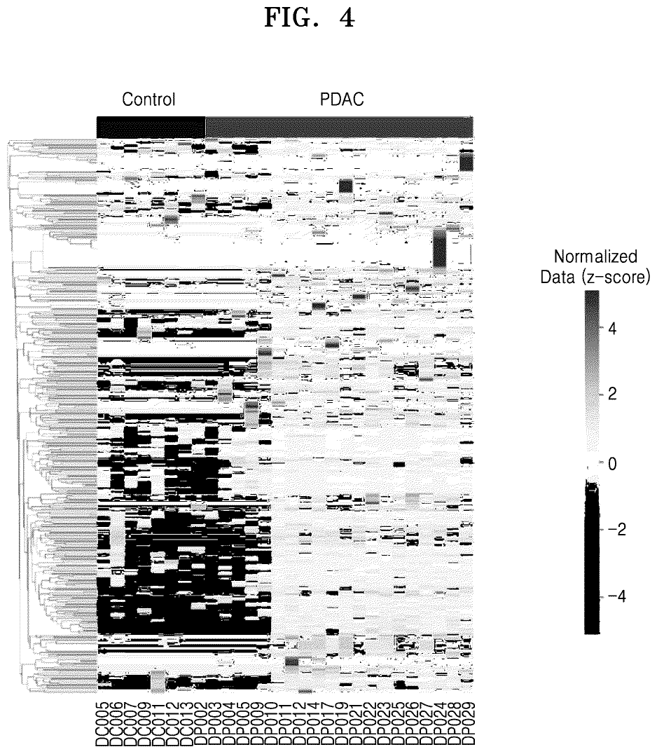

As shown in FIG. 4, expression of a total of 430 miRNAs was identified in exosomes derived from duodenal-pancreatic fluid from eight control subjects (Control) and twenty pancreatic cancer patients (PDAC), and the expression patterns of all identified miRNAs were presented in a heatmap.

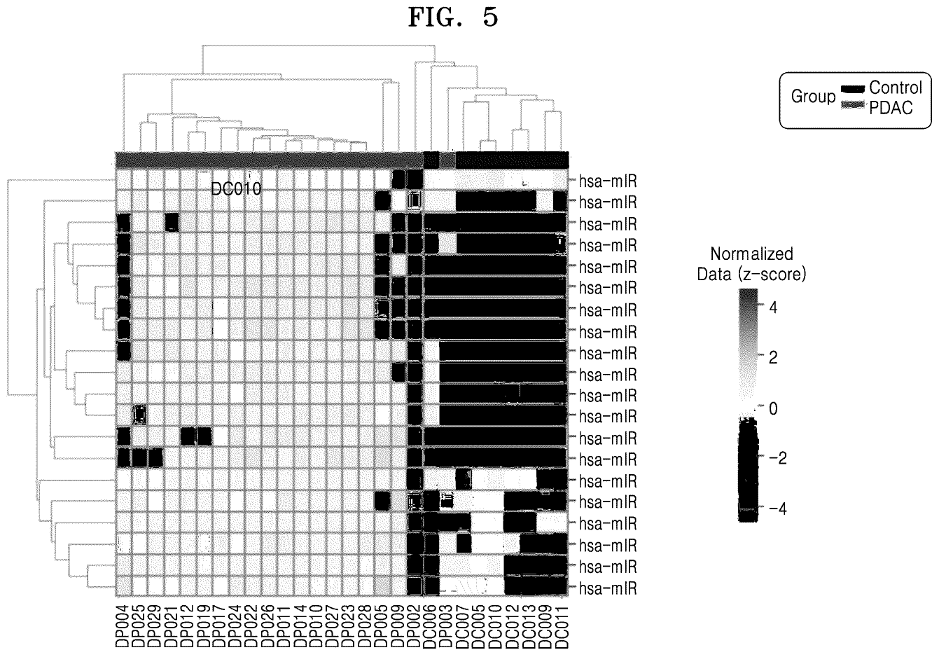

As shown in FIG. 5, a statistical analysis was performed for in-depth examination of miRNA sequencing data from exosomes derived from duodenal-pancreatic fluid.

Specifically, based on the expression data of all 430 miRNAs identified in the heatmap, a Student's t-test was performed to identify miRNAs showing significant increases or decreases in the pancreatic cancer group compared to the normal control group, thereby identifying miRNA candidates with statistically significant changes.

Twenty candidate miRNAs meeting the criteria of a 2-fold change, Log 2>2, and Pval<0.01 were identified from the analysis data. These miRNAs showed a marked increase in the pancreatic cancer group compared to the control group and were significantly elevated in all pancreatic cancer patients.

### Example 4. Correlation Matrix Analysis Based on Individual Control and Pancreatic Cancer Group Sequencing Data

A correlation matrix analysis was performed by comparing the individual sequencing data of eight control subjects and twenty pancreatic cancer patients described in Example 3, focusing on the similarity among samples to identify specific expression patterns in each group.

Specifically, exosome-derived miRNA expression data obtained from the control group (8 subjects) and the pancreatic cancer group (20 subjects) were loaded using the Pandas library. Missing values or outliers were identified and removed or corrected, and preprocessed into a format suitable for analysis. To evaluate the correlation among miRNA expressions based on the entire sample data, either the Pearson correlation coefficient or Spearman correlation coefficient was calculated. For this purpose, the NumPy or SciPy libraries were utilized. The calculation of the correlation coefficients was based on the following formulas:
Pearson correlation coefficient: evaluates the linear correlation between two variables; and
Spearman correlation coefficient: evaluates a rank-based correlation, including nonlinear relationships between variables.

As a result, an n×n correlation matrix representing the correlation among miRNAs was generated. Correlation coefficients for each group were also calculated separately by dividing the data into control and pancreatic cancer patient groups. This allowed comparison of differences in miRNA correlation patterns between the two groups. The same calculation method (Pearson or Spearman) was used for this analysis. The resulting correlation matrices were visualized as heatmaps using the Seaborn library. The heatmap colors were set to correspond to the correlation coefficient values (e.g., from −1 to 1), providing an intuitive indication of the strength and direction of the correlation among miRNA expressions. Differences between the correlation matrices for the control and patient groups were analyzed by calculating the difference between the corresponding elements in each matrix. For example, for each miRNA pair, the difference between the correlation coefficients in the control group and those in the patient group was computed, thereby identifying miRNA pairs showing large differences between the two groups. These differences were calculated using NumPy. The analysis results were used to identify miRNA pairs with markedly altered correlations.

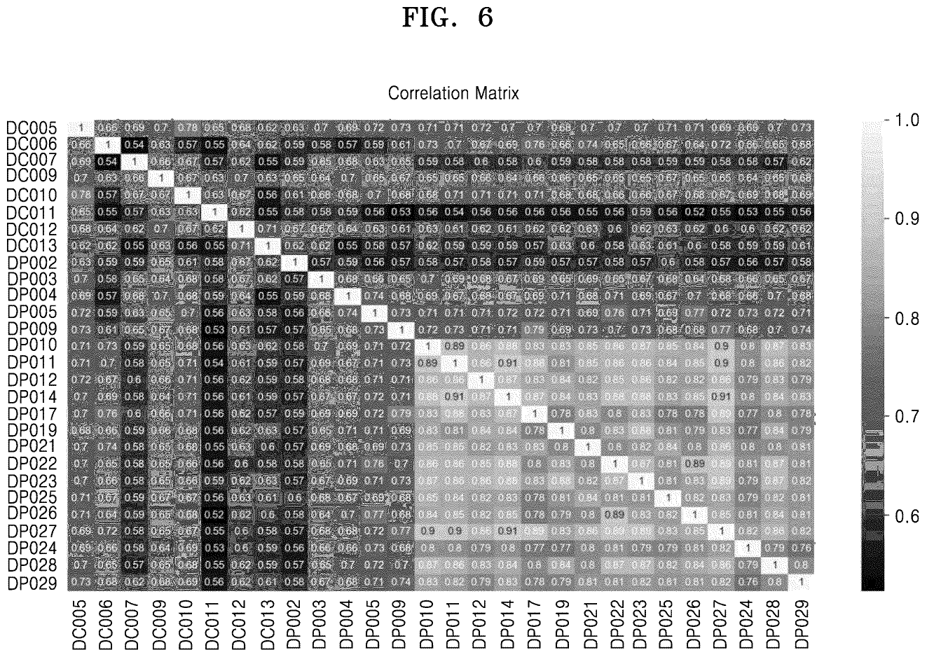

As a result, as shown in FIG. 6, the similarity score within the control group was low, whereas the pancreatic cancer group showed a markedly high similarity score (0.7-0.9) among its individual patient samples (see the orange portion in FIG. 6).

These findings indicate that, compared to the control group, the pancreatic cancer group showed a higher tendency toward within-group similarity among individual samples, and that, upon the onset of pancreatic cancer, exosomal miRNAs in duodenal-pancreatic fluid undergo similarly pronounced changes in expression patterns.

### Example 5. Principal Component Analysis (PCA) Based on Sequencing Data from Control Group and Pancreatic Cancer Group

Using the individual sequencing data from eight control subjects and twenty pancreatic cancer patients described in Example 3, the expression patterns of each miRNA were analyzed, and a three-dimensional PCA analysis was performed using three reference points (PC1, PC2, and PC3) based on similarity.

Specifically, exosome-derived miRNA expression data obtained from the control group (8 subjects) and the pancreatic cancer patient group (20 subjects) were analyzed using the Pandas library. The data were preprocessed by removing missing values and unifying the data format so that the same set of miRNAs was retained for all samples. To improve the accuracy of the PCA analysis, the data were subsequently scaled. Using the [StandardScaler] in scikit-learn, each miRNA expression value was standardized to a mean of 0 and a standard deviation of 1 (to prevent differences in miRNA expression units from affecting the PCA results). A PCA model was created using the PCA module of scikit-learn. Three principal components (PCs) were set to be extracted, according to the purpose of the analysis. The model was generated as follows:
the principal components were calculated to explain the maximum possible variance in the data; and
each principal component was composed of a linear combination of the original data and was guaranteed to be orthogonal to the others.

The PCA model was applied to the data to calculate principal component scores for each sample. These scores represented the coordinates of each sample in a new three-dimensional space. The control and patient group data were input into the same PCA model to generate comparable principal component scores. Using Axes3D in Matplotlib and Seaborn, the control and patient data were visualized in three dimensions. Visualization was performed as follows:
plotting each sample using the three principal components (PC1, PC2, PC3) as coordinates;
distinguishing between the control group and the patient group by color or marker style; and
generating a three-dimensional, rotatable graph to intuitively observe the data distribution.

As a result of the PCA analysis, a difference in data distribution between the orange-colored control group and the green-colored patient group was observed. The explained variance ratio of the principal components was analyzed to assess how much each principal component accounts for the variability in the data.

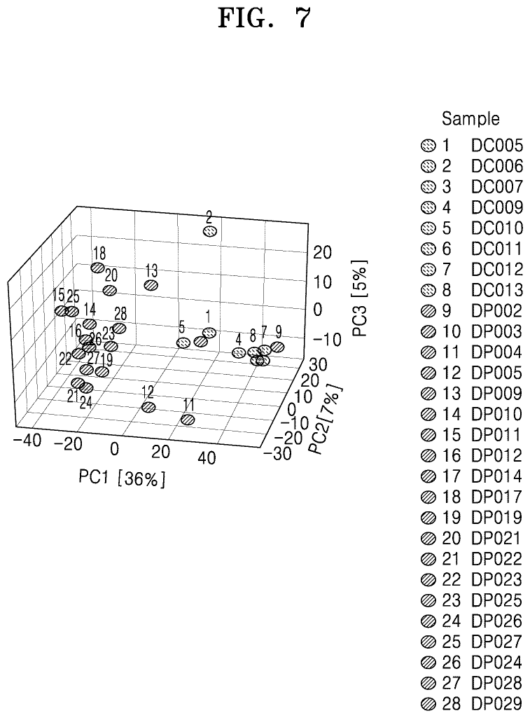

As shown in FIG. 7, the orange-colored control group and the green-colored pancreatic cancer group each exhibited similar expression patterns within their respective groups, with their data points clustering together in the three-dimensional graph.

### Example 6. miRNA Expression Analysis by TNM Stage

To identify biomarkers specifically expressed in early-stage pancreatic cancer patients, group analysis was performed for each TNM stage using a heatmap analysis technique. Specifically, the control group and pancreatic cancer patients in stages 1, 2, and 4 were divided into groups and analyzed individually, and miRNAs meeting the conditions of 2-fold change, Log 2>6, and Pval<0.05 were identified within each group analysis.

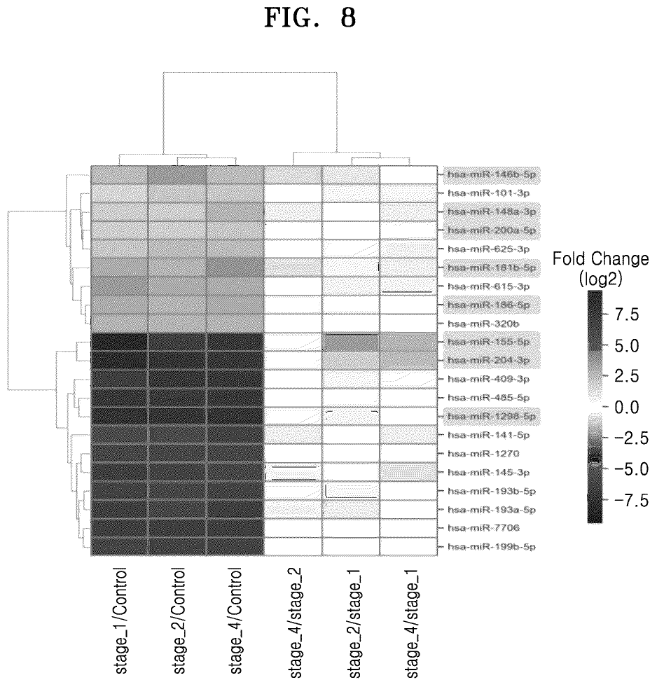

As a result, as shown in FIG. 8, a total of 21 exosomal miRNAs were identified, and all of these miRNAs showed specifically increased expression in all stages of pancreatic cancer compared to the control group. In particular, the miRNA candidates highlighted in red in FIG. 8 were the same miRNA candidates derived from the analysis in Example 3, indicating their greater significance.

Furthermore, miRNAs showing stage-specific expression were analyzed by comparing the fold change values between each stage group of pancreatic cancer patients and the control group.

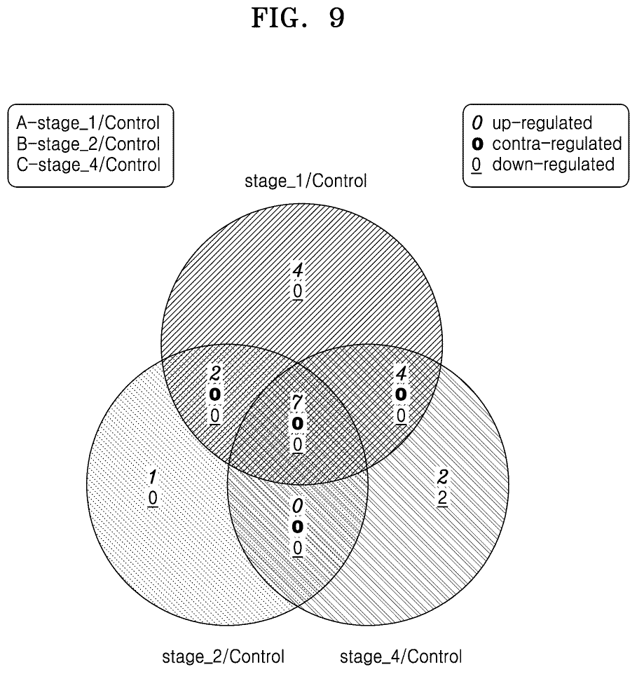

As a result, as shown in FIG. 9, miRNA candidates showing specific expression changes in stage 1 pancreatic cancer patients (miR-155-5p, miR-423-5p) were identified, along with miRNAs that showed increased expression in all pancreatic cancer stages (miR-101-3p, miR-148a-3p, miR-1298-5p) and miRNAs showing expression changes specific to late-stage pancreatic cancer patients (miR-200a-5p, miR-30e-3p).

### Example 7. Analysis of Specificity and Sensitivity of Pancreatic Cancer-Specific miRNAs

An area under the curve (AUC) analysis was performed to assess the diagnostic specificity and sensitivity of eight candidate miRNAs-out of the twelve pancreatic cancer-specific exosomal miRNAs derived from duodenal-pancreatic fluid-that showed increased expression in the pancreatic cancer group. Similarly, graphs were constructed based on the expression levels of each candidate miRNA in exosomes obtained from the duodenal-pancreatic fluid of the control group (control) and the pancreatic cancer group (PDAC), and an AUC analysis was subsequently performed.

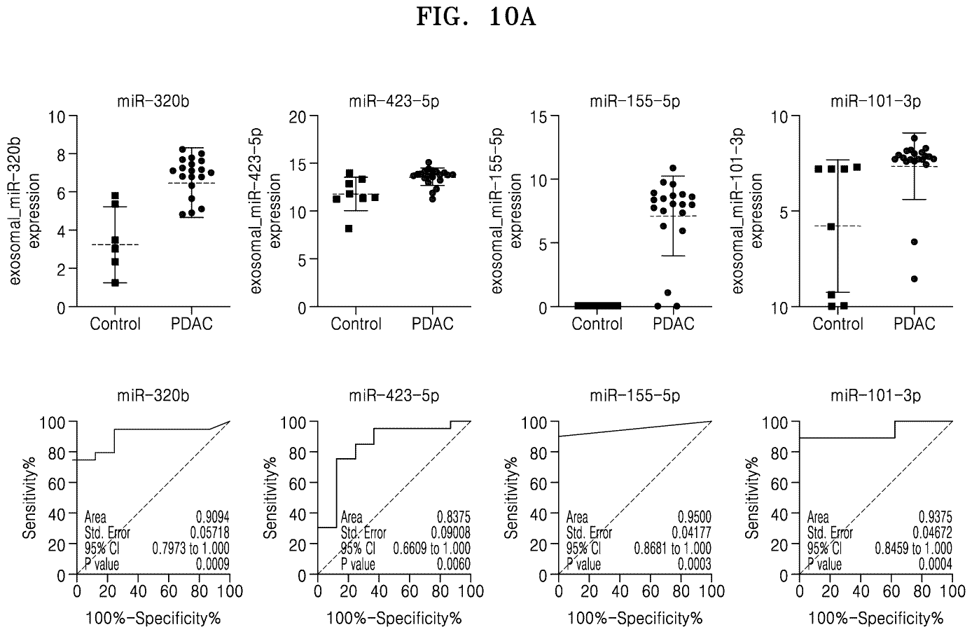

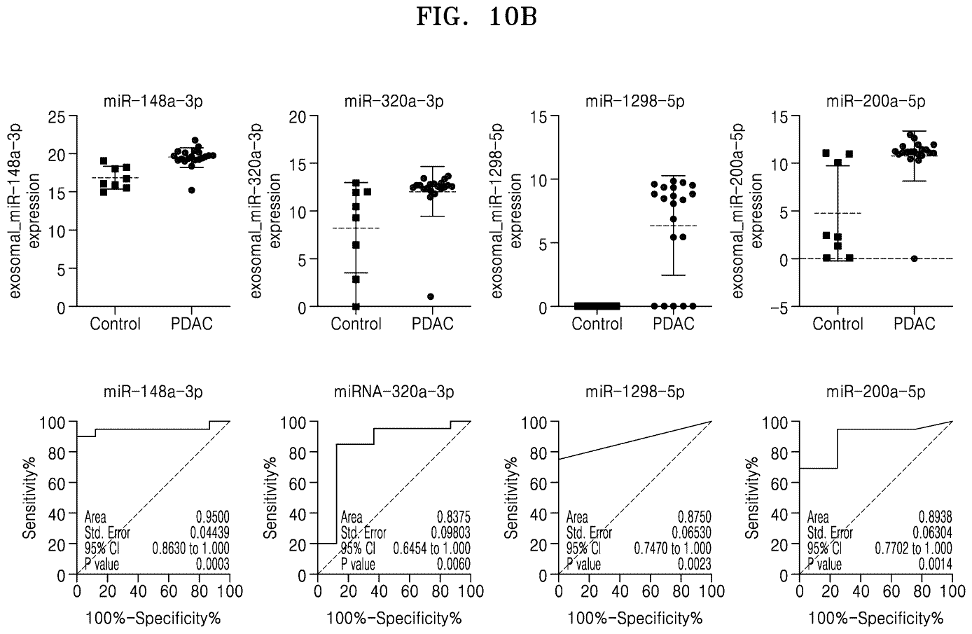

As shown in FIGS. 10A and 10B, the results confirmed the following AUC values: miR-320b showed an AUC of 0.90 (95% Cl, 0.7973 to 1), miR-423-5p showed an AUC of 0.83 (95% Cl, 0.6609 to 1), miR-155-5p showed an AUC of 0.95 (95% Cl, 0.8681 to 1), miR-101-3p showed an AUC of 0.94 (95% Cl, 0.8459 to 1), miR-148a-3p showed an AUC of 0.95 (95% Cl, 0.8630 to 1), miR-320a-3p showed an AUC of 0.84 (95% Cl, 0.6454 to 1), miR-1298-5p showed an AUC of 0.87 (95% Cl, 0.7470 to 1), and miR-200a-5p showed an AUC of 0.89 (95% Cl, 0.7702 to 1). These findings demonstrated that all eight candidate miRNAs are significant biomarkers for diagnosing pancreatic cancer.

Additionally, an AUC analysis was performed to assess the diagnostic specificity and sensitivity of four candidate miRNAs-out of the twelve pancreatic cancer-specific exosomal miRNAs derived from duodenal-pancreatic fluid-that showed decreased expression in the pancreatic cancer group. Graphs were constructed based on the expression levels of each candidate miRNA in exosomes obtained from the duodenal-pancreatic fluid of the control group (control) and the pancreatic cancer group (PDAC), and an AUC analysis was subsequently performed.

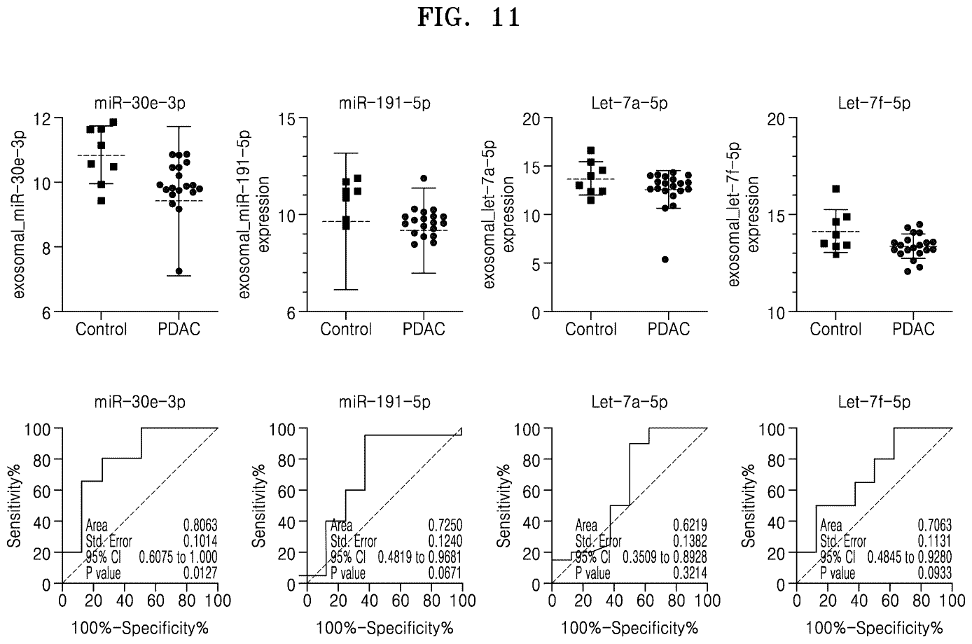

As shown in FIG. 11, miR-30e-3p showed an AUC of 0.8 (95% Cl, 0.6075 to 1), miR-191-5p showed an AUC of 0.72 (95% Cl, 0.4819 to 0.9681), Let-7a-5p showed an AUC of 0.62 (95% Cl, 0.3509 to 0.8928), and Let-7f-5p showed an AUC of 0.70 (95% Cl, 0.4845 to 0.928). These findings demonstrated that all four candidate miRNAs are significant biomarkers for diagnosing pancreatic cancer.

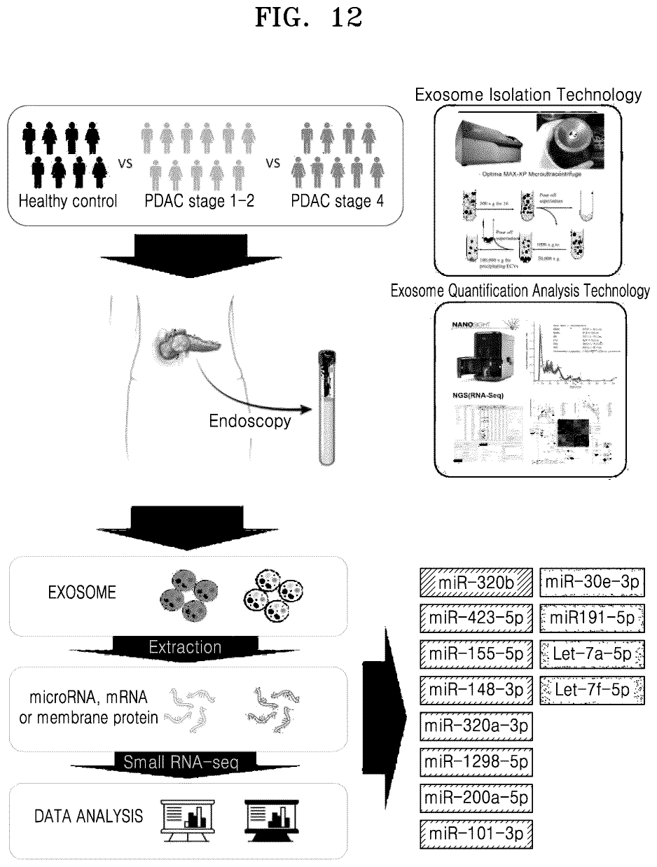

In conclusion, in the present disclosure, exosomes isolated from the duodenal-pancreatic fluid of normal controls and pancreatic cancer patients were used for small RNA sequencing, and statistical as well as bioinformatics methods were applied to the resulting sequencing data, ultimately identifying twelve duodenal-pancreatic-fluid-derived exosomal miRNAs that are specific to pancreatic cancer (see FIG. 12). These identified miRNAs exhibit excellent diagnostic specificity and sensitivity for pancreatic cancer and thus can be usefully employed for non-invasive diagnosis of pancreatic cancer and for predicting the progression and treatment response of pancreatic cancer.

According to an aspect, the exosome-derived miRNAs isolated from duodenal-pancreatic fluid exhibit excellent diagnostic specificity and sensitivity for pancreatic cancer and can be employed in a non-invasive manner not only to diagnose pancreatic cancer but also to predict progression and treatment response of pancreatic cancer.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. A method for diagnosing pancreatic cancer, the method comprising:
obtaining a biological sample from a subject;
measuring the expression level of at least one miRNA selected from the group consisting of miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p in the biological sample; and
diagnosing pancreatic cancer based on the expression level of the at least one miRNA.

2. The method of claim 1, wherein the biological sample is at least one selected from tissue, cells, whole blood, blood, serum, saliva, sputum, cerebrospinal fluid, urine, and exosomes derived therefrom.

3. The method of claim 1, wherein the miRNA is derived from an exosome isolated from duodenal-pancreatic fluid.

4. The method of claim 1, wherein the measuring the expression level of at least one miRNA is conducted using a composition comprising a reagent capable of measuring an expression level of at least one miRNA selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p and Let-7f-5p.

5. The method of claim 4, wherein the reagent capable of measuring the expression level of at least one miRNA comprises the miRNA or a contiguous polynucleotide of the miRNA or a portion thereof, or a polynucleotide complementary thereto.

6. The method of claim 1, wherein the expression level is measured by at least one selected from next-generation sequencing (NGS), PCR, RT-PCR, real-time PCR, an RNase protection assay (RPA), a microarray, and Northern blotting.

7. The method of claim 1, further comprising comparing the expression level of the at least one miRNA selected from the group consisting of miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p, as measured in the biological sample, to the expression level of the same miRNA measured in a biological sample obtained from a normal control.

8. A method of providing information for diagnosing pancreatic cancer, the method comprising: (a) measuring, in a biological sample isolated from a subject, an expression level of at least one miRNA gene selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p; and
(b) comparing the expression level of the at least one miRNA gene selected from miR-320b, miR-423-5p, miR-155-5p, miR-101-3p, miR-148a-3p, miR-320a-3p, miR-1298-5p, miR-200a-5p, miR-30e-3p, miR-191-5p, Let-7a-5p, and Let-7f-5p as measured in step (a) to an expression level of the same miRNA gene measured in a biological sample isolated from a normal control.

9. The method of claim 8, wherein the biological sample is at least one selected from tissue, cells, whole blood, blood, serum, saliva, sputum, cerebrospinal fluid, urine, and exosomes derived therefrom.

10. The method of claim 8, further comprising (c-1) diagnosing pancreatic cancer when an expression level of at least one miRNA gene selected from miR-320b, miR-423-5p, miR-155-5p, miR-101-3p, miR-148a-3p, miR-320a-3p, miR-1298-5p, and miR-200a-5p, as measured in step (a), is higher than an expression level of the same miRNA gene measured in the biological sample obtained from the normal control in step (b).

11. The method of claim 8, further comprising (c-2) diagnosing pancreatic cancer when an expression level of at least one miRNA gene selected from miR-30e-3p, miR-191-5p, Let-7a-5p, and Let-7f-5p as measured in step (a) is lower than an expression level of the same miRNA gene measured in the biological sample obtained from the normal control in step (b).

12. The method of claim 10, wherein stage I pancreatic cancer is diagnosed when an expression level of miR-155-5p or miR-423-5p gene is higher than an expression level of the same miRNA gene measured in a biological sample isolated from a normal control, and late-stage pancreatic cancer is diagnosed when an expression level of miR-200a-5p gene is higher than an expression level of the same miRNA gene measured in a biological sample isolated from a normal control.

13. The method of claim 11, wherein late-stage pancreatic cancer is diagnosed when an expression level of miR-30e-3p gene is lower than an expression level of the same miRNA gene measured in a biological sample isolated from a normal control.

14. The method of claim 8, wherein the expression level is measured by at least one selected from next-generation sequencing (NGS), PCR, RT-PCR, real-time PCR, an RNase protection assay (RPA), a microarray, and Northern blotting.
